(19) United States
(12) Patent Application Publication
(10) Pub. No.: US 2014/0212388 A1
(43) Pub. Date: Jul. 31, 2014

(54) Composition For Cosmetic And Health Improvement

(71) Applicant: Maria Alvarez Prieto, Chatham, NJ (US)

(72) Inventor: Maria Alvarez Prieto, Chatham, NJ (US)

(21) Appl. No.: 14/157,864

(22) Filed: Jan. 17, 2014

Related U.S. Application Data
(60) Provisional application No. 61/758,512, filed on Jan. 30, 2013.

(51) Int. Cl. A61K 33/00; A61K 31/4172; A61K 31/728; A61K 31/4188

(52) U.S. Cl. CPC A61K 33/00; A61K 31/4188; A61K 31/4172; A61K 31/728
USPC 424/93.4; 424/600

(57) ABSTRACT

The invention relates to compositions in a form of health drinks, capsules, tablets or powders. In particular the compositions improve individual's general health, maintain and improve regeneration of human connective tissue and simultaneously provide positive cosmetic effects for a better life and more happiness for all, male, female, young and old. The composition may include compounds such as silica compound, biotin, carnosine, hyaluronic acid, methylsulfonyl methane (MSM), dimethylethanolamine (DMEA) and omega-3-oil along with other components.

### CLAIM OF PRIORITY

This application claims priority to U.S. Ser. No. 61/758,512 filed Jan. 30, 2013, the contents of which are fully incorporated herein by reference.

### FIELD OF THE INVENTION

The invention relates to health compositions, in particular in form of health drinks, capsules, tablets or powders improving individual's general health, vitality and simultaneously having positive cosmetic effects.

### BACKGROUND OF THE INVENTION

There are various publications disclosing compositions and compounds having a positive effect on the overall health and vitality of an individual. Also there are various publications disclosing cosmetic compositions and compounds improving condition of one's skin. There are a number of publications disclosing compositions for apical application to improve the health of the skin.

U.S. Pat. No. 8,192,748 discloses a cosmetic composition for apical application on the skin.

US patent application number 2007/0140984 discloses a powdery product for use as an external dermatological agent.

US patent application number 2007/0166255 discloses a composition for treatment of topical discomforts.

On the other hand there are publications disclosing various orally administered compositions for treating and alleviating syndromes of joint inflammation and pain and other health conditions. An example is the U.S. Pat. No. 6,924,273 providing an orally administered composition comprising hyaluronic acid to treat osteoarthritis, joint effusion and other conditions.

Various compounds and compositions are generally known to have positive health effects and some ingredients are used as dietary supplements:

Silicon dioxide, also known as silica has the chemical formula of SiO₂. Silicon is known to stimulate cell metabolism and cell formation; it helps prevent the aging process on tissues and is important for the structure and function of connective tissue. Silica is known to increase elasticity and firmness of blood vessels. It is also known as an anti-inflammatory, disinfecting, absorbing and odor binding agent.

Hyaluronic acid is a substance naturally present in the human body. It is found in the highest concentrations in fluids in the eyes and joints. Hyaluronic acid is usually taken for various joint disorders, including osteoarthritis.

Biotin is necessary for cell growth, for production of fatty acids, and for metabolism of fats and amino acids. Biotin is also known as Vitamin H and Vitamin B7. Biotin has been reported to be helpful in maintaining a steady blood sugar level. It is used as a dietary supplement for strengthening hair and nails. http://en.wikipedia.org/wiki/Biotin-cite_note-4

Chondroitin is usually used together with glucosamine to alleviate syndromes of osteoarthritis.

Methylsulfonylmethane (MSM) is an organosulfur compound with the formula (CH₃)₂50₂. It is also known by several other names including DMSO₂, methyl sulfone, and dimethyl sulfone. MSN is used as a dietary supplement often in combination with glucosamine and chondroitin for treatment or prevention of osteoarthritis.

Accordingly, there are various publications providing compounds for topical use to improve skin health, and to provide cosmetic advantages. On the other hand there are orally administered compositions to improve joint health, bone health, and to improve growth of hair and nails.

The general elements of the contemporary life style include stress, lack of sleep, less than perfect nutrition, and air pollution among other features. All these elements are negatively affecting the overall health of individuals, and among other symptoms are various skin problems, including dryness, tiredness, and lack of radiance, and so on.

The instant invention provides a health drink that includes essential elements to improve the overall health, to provide a good sleep, reduce stress and to improve the health of the skin.

### SUMMARY OF THE INVENTION

It is an object of this invention to provide a composition for oral administration to improve overall health and well being.

It is another object of this invention to provide a composition for oral administration to improve skin health.

Yet another object of this invention is to provide a composition for oral administration to improve regeneration of connective tissue, including among others skin, joints, tendons, and ligaments, muscle tissue, gum tissue, hair, and nails, as well as suspension of every organ and tissue in the body.

It is yet an object of this invention to provide a composition in form of a health drink, capsule, tablet or powder having positive effects on overall health and on skin smoothness, moisture and radiance.

It is an object of this invention to provide a composition and a health drink improving general health and providing positive cosmetic effects and comprising one or more ingredients selected from the group consisting of: silica compound, biotin, hyaluronic acid, methylsulfonyl methane (MSM), dimethylethanolamine (DMEA) and omega-3-oil.

It is an objective of this invention to provide a health drink comprising: a silica compound, biotin, hyaluronic acid, methylsulfonyl methane (MSM), dimethylethanolamine (DMEA), omega-3-oil, and at least one additional ingredient selected from the group consisting of: Oregano (Origanum vulgare), Hawthorn (Crataegus sp.), Cardamom (Elettaria cardamomum), Cinnamon (Cinnamomum verum), Turmeric (Curcuma longa), Holy basil (Ocimum tenuiflorum), Soybeans (Glycine max), Gingerroot (Zingiber officinale), Ginko biloba, Rosemary (Rosmarinus officinalis), Red yeast rice (rice kernels with Monascus purpureus colonies), Lions Mane mushroom (Hericium erinaceus), Schitzonepeta sp., Gotu Kola (Centiella asiatica), Bacoba monnieri, onion extract, garlic extract, olive leaf extract, artichoke leaf extract, Moomiyo extract, Vitamin A, Vitamin B3, Vitamin B5, Vitamin B6, Vitamin B12; vitamin C, vitamin D3, Vitamin K, Vitamin K2, and Vitamin E, alpha-ketoglutarate choline, cytinediphosphate (CDP)-choline, L-alpha-glyserophosphocholine, phosphathidyl choline, tocopherols, tocotrienols, carotenoids, curcumin, reduced form of NADP (nicotinamide adenine dinucleotide phosphate); DHEA (dehydroepiandrosterone), pregnenolone, resveratrol, lechitin, triple lechitin, high choline lechitin, glucosamine sulfate, condroitin sulfate, omega-3-fatty acids, probiotics, huperzine A, and ubiquinol.

### DESCRIPTION OF THE PREFERRED EMBODIMENTS

The preferred embodiments of the present invention will now be described.

According to a preferred embodiment of the instant invention, a composition for oral administration improving health and having positive cosmetic effects is provided, said composition comprising a silica compound, biotin, carnosine, and hyaluronic acid.

According to another preferred embodiment the composition comprises a silica compound, biotin, carnosine, hyaluronic acid, methylsulfonyl methane (MSM), dimethylethanolamine (DMEA), and omega-3-oil.

According to yet another preferred embodiment the composition comprises a silica compound, biotin, carnosine, hyaluronic acid, methylsulfonyl methane (MSM), dimethylethanolamine (DMEA), omega-3-oil, and one or more additional ingredients selected from the group consisting of vitamins, minerals, plant extracts, natural plant or fungal ingredients, amino acids and their derivatives, hormones, antioxidants and other components.

The composition may be a drink or it may be in concentrated formula or powder for making a health drink.

The composition may also be administered as a tablet, capsule or powder.

### DETAILED DESCRIPTION OF THE INVENTION

The composition of this invention is meant to improve the general health of the user. The composition improves hair growth, and skin health. It improves healthy blood pressure, lowers stress, improves the quality of night sleep and reduces symptoms of arthritis and other joint pains, and increases mobility and flexibility.

The composition of this invention improves happiness and well being of the users by preventing and delaying signs of aging. The composition delays signs of aging in young people and in middle aged and older people it enhances ability to move and be active, to look younger and have more fulfilling life.

The composition of this invention improves health of connective tissue. The composition prevents and delays signs of aging of the connective tissue. The composition improves regeneration of human connective tissue, prevents sagging of skin, maintains elasticity and integrity of ligaments, tendons, and muscle tissue, as well as vascular tissue, hair, nails, gum tissue and sustains every organ of the body to maintain youthful body shape inside and out. The composition also maintains healthy joints and bones.

The composition of this invention is preferably provided as a health drink, although the composition may be provided as a tablet, capsule or a powder as well. The drink may be carbonated or non-carbonated. Alternatively, the composition is provided as a concentrate that is diluted with water. According to yet another embodiment the composition is provided as a powder or a tablet or capsule that is dissolved into water when desired to be used. The composition of this invention may be used as a drink that can be enjoyed daily.

An essentially dry mixture of the beverage can be prepared by blending the proper amounts and ratios of all the required dry ingredients together. The beverage can be provided as a health drink or as a concentrate that is to be diluted with water. Alternatively, the finally prepared drinkable beverage composition can be dehydrated to give the essentially dry mixture of the beverage composition. An edible binding agent, such as starch, can be added to a powder form of the beverage composition and the resulting mixture compacted into tablets. The essentially dry mixture, either as powder, granules or tablets, can later be dissolved in a proper amount of water, carbonated or noncarbonated, to make the final drinkable beverage.

The essential ingredients in the beverage, the concentrate or essentially dry powder or tablet are a silica compound, biotin and hyaluronic acid. A preferred embodiment also contains methylsulfonylmethane (MSM), dimethylethanolamine (DMEA) and omega-3-oil. One or more ingredients selected from the group consisting of vitamins, minerals, plant extracts, natural plant or fungal ingredients, amino acids and their derivatives, hormones, choline-compounds, antioxidants, and other components may be included into the composition.

Vitamins may be selected from the group consisting of: Vitamin A, Vitamin B3, Vitamin B5, Vitamin B6, Vitamin B12; vitamin C, vitamin D3, Vitamin K, Vitamin K2 and Vitamin E.

Minerals may be selected form the group consisting of: chromium, zinc, copper, magnesium, calcium, and selenium.

Plant extracts may be selected from the group consisting of: onion extract, garlic extract, olive leaf extract, artichoke leaf extract; and Moomiyo-extract.

Plant and fungal ingredients are selected from the group consisting of: Oregano (Origanum vulgare), Hawthorn (Crataegus sp.), Cardamom (Elettaria cardamomum), Cinnamon (Cinnamomum verum), Turmeric (Curcuma longa), Holy basil (Ocimum tenuiflorum), Soybeans (Glycine max), Gingerroot (Zingiber officinale), Ginko biloba, Rosemary (Rosmarinus officinalis), Bacoba monnieri, Red yeast rice (rice kernels with Monascus purpureus colonies), Lions Mane mushroom (Hericium erinaceus), Schitzonepeta sp., and Gotu Kola (Centiella asiatica)

Hormones are selected from the group consisting of: DHEA (dehydroepiandrosterone) and pregnenolone.

Amino acids and their derivatives are selected from the group consisting of: glycine, glutamate, L-arginine; L-arginine-HCl, L-glutamine, L-DOPA (L-3,4-dihydroxyphenylalanine), L-isoleucine; L-proline, L-tyrosine, L-valine; L-lysine, L-lycine-HCl, Oxo-proline, N-acetyl cysteine, L-carnosine, Carnitine, L-carnitine, Acetyl-L-carnitine, ornithine alphaketoglutarate, and phosphatidyl serine.

Choline compounds may be selected from the group consisting of alpha-ketoglutarate choline, cytinediphosphate (CDP)-choline, L-alpha-glyserophosphocholine, and phosphathidyl choline.

Antioxidants may be selected from the group consisting of tocopherols, tocotrienols, carotenoids, and ascorbic acid.

Other ingredients may include curcumin, reduced form of NADP (nicotinamide adenine dinucleotide phosphate), resveratrol, lechitin, triple lechitin, high choline lechitin, phosphatidyl serine, gamma amino-butyuric acid (GABA), glucosamine sulfate, chondroitin sulfate, omega-3-fatty acids, probiotics, huperzine A, Pycnogenol®, or ubiquinol.

One or more of the ingredients described above may be added to the essential ingredients to make the beverage, the concentrate or the essentially dry powder or tablet. A drink may be prepared with or without carbon dioxide. A preservative may be used with the drink but is generally not required in the concentrate or, in particular, in the essentially dry powder or tablet.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A composition improving general health and providing positive cosmetic effects, said composition comprising one or more ingredients selected from the group consisting of: silica compound, biotin, carnosine, hyaluronic acid methylsulfonyl methane (MSM), dimethylethanolamine (DMEA) and omega-3-oil.

2. The composition of claim 1, wherein the composition additionally comprises one or more compounds selected from the group consisting of natural plant or fungal ingredients, plant extracts, minerals, vitamins, hormones, antioxidants, choline compounds and amino acids or their derivatives.

3. A composition improving general health and providing positive cosmetic effects, said composition comprising silica compound, biotin, carnosine and hyaluronic acid.

4. The composition of claim 3, wherein the composition additionally comprises methylsulfonyl methane (MSM), dimethylethanolamine (DMEA) and omega-3-oil.

5. The composition of claim 4, wherein the composition additionally comprises one or more compounds selected from the group consisting of natural plant or fungal ingredients, plant extracts, minerals, vitamins, hormones, antioxidants, choline compounds and amino acids or their derivatives.

6. The composition of claim 5, wherein the natural plant or fungal ingredients are selected from the group consisting of: Oregano (Origanum vulgare), Hawthorn (Crataegus sp.), Cardamom (Elettaria cardamomum), Cinnamon (Cinnamomum verum), Turmeric (Curcuma longa), Holy basil (Ocimum tenuiflorum), Soybeans (Glycine max), Gingerroot (Zingiber officinale), Ginko biloba, Rosemary (Rosmarinus officinalis), Bacoba monnieri, Red yeast rice (rice kernels with Monascus purpureus colonies), Lions Mane mushroom (Hericium erinaceus), Schitzonepeta sp., and Gotu Kola (Centiella asiatica).

7. The composition of claim 5, wherein the plant extracts are selected from the group consisting of: onion extract, garlic extract, olive leaf extract, artichoke leaf extract, and Moomiyo extract.

8. The composition of claim 5, wherein the minerals are selected from the group consisting of: chromium, zinc, copper, magnesium, calcium, and selenium.

9. The composition of claim 5, wherein the vitamins are selected from the group consisting of: Vitamin A, Vitamin B3, Vitamin B5, Vitamin B6, Vitamin B12; vitamin C, vitamin D3, Vitamin K, Vitamin K2, and Vitamin E.

10. The composition of claim 5, wherein the amino acids and their derivates are selected from the group consisting of: glycine, glutamate, L-arginine; L-arginine-HCl, L-glutamine, L-DOPA (L-3,4-dihydroxyphenylalanine), L-isoleucine; L-proline, L-tyrosine, L-valine; L-lysine, L-lycine-HCl, Oxo-proline, N-acetyl cysteine, L-carnosine, Carnitine, L-carnitine, Acetyl-L-carnitine, ornithine alphaketoglutarate, and phosphatidyl serine.

11. The composition of claim 5, wherein the hormones are selected from the group consisting of DHEA (dehydroepiandrosterone) and pregnenolone.

12. The composition of claim 5, wherein the choline compounds are selected from the group consisting of alpha-ketoglutarate choline, cytinediphosphate (CDP)-choline, L-alpha-glyserophosphocholine, and phosphathidyl choline.

13. The composition of claim 5, wherein the antioxidants are selected from the group consisting of: tocopherols, tocotrienols, carotenoids, and ascorbic acid.

14. The composition of claim 5, wherein the composition additionally includes ingredients selected from: curcumin, reduced form of NADP (nicotinamide adenine dinucleotide phosphate); DHEA (dehydroepiandrosterone), pregnenolone, resveratrol, lechitin, triple lecthitin, high choline lechitin, glucosamine sulfate, condroitin sulfate, omega-3-fatty acids, probiotics, huperzine A, Pycnogenol®, and ubiquinol.

15. The composition of claim 5, wherein the composition is a drink.

16. The composition of claim 5, wherein the composition is a powder or a tablet soluble to water.

17. An oral composition comprising: a silica compound, biotin, hyaluronic acid, methylsulfonyl methane (MSM), dimethylethanolamine (DMEA), omega-3-oil, and at least one additional ingredient selected from the group consisting of: Oregano (Origanum vulgare), Hawthorn (Crataegus sp.), Cardamom (Elettaria cardamomum), Cinnamon (Cinnamomum verum), Turmeric (Curcuma longa), Holy basil (Ocimum tenuiflorum), Soybeans (Glycine max), Gingerroot (Zingiber officinale), Ginko biloba, Rosemary (Rosmarinus officinalis), Red yeast rice (rice kernels with Monascus purpureus colonies), Lions Mane mushroom (Hericium erinaceus), Schitzonepeta sp., Gotu Kola (Centiella asiatica), onion extract, garlic extract, olive leaf extract, artichoke leaf extract, Moomiyo extract, Vitamin A, Vitamin B3, Vitamin B5, Vitamin B6, Vitamin B12; vitamin C, vitamin D3, Vitamin K, Vitamin K2, and Vitamin E, alpha-ketoglutarate choline, cytinediphosphate (CDP)-choline, L-alpha-glyserophosphocholine, phosphathidyl choline, tocopherols, tocotrienols, carotenoids, curcumin, reduced form of NADP (nicotinamide adenine dinucleotide phosphate); DHEA (dehydroepiandrosterone), pregnenolone, resveratrol, lechitin, triple lecthitin, high choline lechitin, glucosamine sulfate, condroitin sulfate, omega-3-fatty acids, probiotics, huperzine A, Pycnogenol®, and ubiquinol.

18. A method to improve individual's vitality and general health, said method comprising a step of administering orally a composition comprising one or more ingredients selected from the group consisting of silica compound, biotin, carnosine, hyaluronic acid, methylsulfonyl methane (MSM), dimethylethanolamine (DMEA) and omega-3-oil.

19. The method of claim 18, wherein the composition is a drink, capsule, tablet or powder and further comprises on or more compounds selected from the group consisting of natural plant or fungal ingredients, plant extracts, minerals, vitamins, hormones, antioxidants, choline compounds and amino acids or their derivatives.

20. A method to increase individual's general health, improve the regeneration of human connective tissue and provide positive cosmetic effects, as well as delay degeneration of muscle tissue, tendons, ligaments, gum-tissue, hair, nails, joints, skeletal and vascular systems, as well as support and sustain every organ of human body, said method including a step of providing the composition of claim 17.
